(19) United States
(12) Patent Application Publication
(10) Pub. No.: US 2026/0207539 A1
(43) Pub. Date: Jul. 23, 2026

(54) PHARMACEUTICAL KIT FOR PREPARATION OF READY-TO-USE VIGABATRIN SOLUTION

(71) Applicant: Shanghai Aucta Pharmaceuticals Co., Ltd., Shanghai (CN)

(72) Inventors: Zhongqin Wang, Shanghai (CN); Shoufeng Li, Piscataway, NJ (US); Ye Huang, Piscataway, NJ (US); Weilong Bai, Shanghai (CN)

(73) Assignee: Shanghai Aucta Pharmaceuticals Co., Ltd., Shanghai (CN)

(21) Appl. No.: 19/033,064

(22) Filed: Jan. 21, 2025

(51) Int. Cl. A61K 31/197; A61K 9/19; A61K 47/12; A61K 47/14

(52) U.S. Cl. CPC A61K 31/197; A61K 9/19; A61K 47/12; A61K 47/14

(57) ABSTRACT

Disclosed herein is a kit for storing a solid form of vigabatrin or a pharmaceutically acceptable salt thereof and reconstituting it to a solution suitable for multiple uses. Also disclosed are methods of reconstituting the solution and treating a disease.

### FIELD OF INVENTION

The present invention pertains to a novel kit containing vigabatrin in solid form, designed for easy reconstitution into a solution for multiple uses. The kit ensures superior product stability and precise dosing.

### BACKGROUND

Vigabatrin is a commercial product marketed in United States with the trademark Sabril® which is first developed by Lundbeck LLC and indicated for Refractory Complex Partial Seizures and Infantile Spasms. Sabril® currently only launch two dosage forms, oral tablet and powder for oral solution in the market. Only the Sabril® powder can be used in the pediatrics and infants due to the characteristics of this patient population and the need for dose adjustment. Nevertheless, Sabril® powder for oral solution is for single use, and it needs to be mixed with water each time to result in 50 mg/ml solution prior to administration. This may cause medication error, dosage waste, or incorrect dose due to wrong solution concentration prepared by non-medical professionals such as patients and caregivers themselves. Any dosing error can be fatal for vigabatrin as it is a drug marketed with REMS due to its potential impact on vision impairment. The waste in dosage is also a major concern due to health economic reasons and the fact some patients might save the unused powder or solution for future use, which is not supported by current approved label. Although another recently approved commercial product Vigafyde(Vigabatrin) oral solution can be used for multiple uses, that need to train patients or healthcare providers before the first dosing and strictly follow the label; otherwise, medication errors could happen due to different dosing concentrations of 100 mg/ml. As vigabatrin powder for oral solution, 500 mg have been launched for more than 10 years, a lot of patients have been used to the dosing regimen based on resultant solution concentration of 50 mg/ml from vigabatrin powder. The dosing regimen/volume on the basis of such solution concentration have been more than 10 years. It is also a long-term medication, the patients have been used to this dosing habit. The sudden switch to a double concentration medication could lead to overdose if healthcare providers forget to adjust the dosing volume, and this potential medication risk could be fatal due to this black box labeled drug. A need exists for safe and manageable vigabatrin product.

### BRIEF SUMMARY OF THE INVENTION

The multiple dose, ready-to-use vigabatrin product of the present invention mitigates the risk of dosing error and improves patient compliance and dosing accuracy. In addition, for seizure patients, particularly infants or children experiencing seizures or spasms that require long-term treatment, a stable drug delivery vehicle with accurate dosing and a palatable taste for multiple uses is crucial for achieving the desired therapeutic effect.

The present invention relates to a vigabatrin solid composition and a desirable vehicle for reconstitution into ready-to-use oral solution. The Vigabatrin blends and the vehicle are packed in separate containers. The solid form vigabatrin can be readily reconstituted into oral solution when ready for dosing and supplied with, for example, a pressed-in-bottle-adaptor and two dosing syringes. The vigabatrin blends include vigabatrin with or without excipients contained in a bottle. When without any excipients, the blends can consist of vigabatrin or its salt thereof. The vehicle includes quantum sufficit (qs) water and optionally at least one of a sweetener, a preservative, a buffering agent, a flavoring agent, a solubilizer, an antioxidant, a surfactant, an antifoaming agent, a chelating agent, a suspending agent, and a coloring agent or a combination thereof.

The multiple uses of the solution for vigabatrin of 50 mg/ml for a prolonged period of time is achieved without undesirable level of API degradation and supplied kit could deliver repeatable and accurate dosing at different dosing volume.

The reconstituted solution is generally colorless to light yellow and clear that have a favorable taste. The pH range and the concentration are designed for multiple uses.

Also provided is a kit including a solid form vigabatrin and a vehicle for storing the active ingredient and reconstituting it into a solution. Meanwhile the kits can also be used for the dosing and medication. The kit allows for longer term storage of the active ingredient as a stable dry powder for the preparation of a solution comprising vigabatrin or its pharmaceutically equivalent derivative that has a good chemical stability.

Another aspect provides a method for reconstituting the solid form active ingredient into a solution. In some embodiments, the buffer and preservative are added separately into the vehicle.

Another aspect provides a method of treating a CNS disease comprising administering to a subject in need thereof a solution reconstituted from the solid active ingredient described herein.

### DETAILED DESCRIPTION OF THE INVENTION

This present invention relates to a solid dosage composition and a kit for reconstituting a solid form active ingredient into a solution. The solid dosage composition having an active pharmaceutical ingredient containing vigabatrin or a pharmaceutically acceptable salt, isomer, complex, polymorph, hydrate, or ester thereof in a solid form, which can be reconstituted into a solution with a liquid vehicle. The vehicle is the aqueous medium, optionally comprising at least one excipients selected from a sweetener, a preservative, a buffering agent, a flavoring agent, a solubility, an antioxidant, an surfactant, an antifoaming agent, a chelating agent, a suspending agent, and a coloring agent or a combination thereof.

The reconstituted oral solution is suitable for multiple uses in treating CNS diseases such as resistant epilepsy, complex partial seizures, secondary generalized seizures, refractory complex partial seizure, and infantile spasms, for patients including pediatrics and infants. The composition and the solution for multiple uses bring convenience to healthcare provider, improve patient compliance, and reduce human error during dosing of medication. They also enhance the medication safety for pediatrics and infants.

While the following text may reference or exemplify specific embodiments of a composition, an oral solution or a method of treating a disease, it is not intended to limit the scope of the composition, solution or method to such particular reference or examples. Various modifications may be made by those skilled in the art, in view of practical and economic considerations, such as the excipients of the composition and the interval of the administration of the solution for treating or preventing a disease or condition.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “about” and the like as used herein, when used in connection with a numerical variable, generally means that the value of the variable and all values of the variable are within the range of experimental error (e.g., within the 95% confidence interval for the mean) or within +10% or within ±5% of the indicated value.

The term “container” as used herein refers to a bottle could contain a suitable amount of liquid, such as for instance 5 ml, 10 ml, 20 ml, 50 ml, 100 ml, 200 ml, 500 ml, and 1000 ml or any volume within them. The bottle texture could be selected from HDPE, glass, LDPE, PET, and PP.

The term “sweetener” as used herein refers to usual taste-masking agents used in the preparation of pharmaceutical products, such as for instance sucrose, glucose, sorbitol, sucralose, aspartame, saccharin sodium and any other pharmaceutically acceptable sweetener or combination thereof.

The term “flavor” as used herein refers to usual flavoring agents used in the preparation of pharmaceutical products. Nonlimiting examples include peppermint, menthol, cherry, orange, lemon, grenadine flavor, berry flavor, strawberry flavor, banana flavor, orange flavor and peppermint flavor, other acceptable fruit flavors, or their mixtures.

The term “multiple uses” as used herein refers to administering to a subject in need thereof a single solution in portions. There is an interval ranging for instance from about an hour to about 2 months, between the administration of two consecutive portions.

The term “preservative” as used herein refers to usual preservatives used in the preparation of pharmaceutical products, such as antimicrobial agents and chelating agents. Examples of preservatives include, but are not limited to, benzalkonium chloride, cetalokonium chloride, benzoates (e.g. sodium benzoates), benzyl alcohol, methyl paraben, propyl paraben, alkyl gallates, hydroxybenzoates and salts thereof (e.g. methyl or propyl hydroxybenzoates and salts thereof), phenyl mercuric salts (e.g. borates or nitrates), sodium hypocholorite, paraben, potassium sorbate, sodium benzoate, and acetic acid, organic solvent such as ethanol, benzyl alcohol, bronopol, chlorbutol, propylene glycol or other preservative systems.

The term “solid dosage” as used herein refers to a dosage of solid state or a solid form of an active ingredient. Non-limiting examples include powder, granules, flakes, spheroids and other forms, which can be readily prepared into a desired solution when added to an ingestible liquid. In some embodiments, the solid dosage is dry and flowable.

The term “therapeutically effective amount” refers to an amount of a compound or an active pharmaceutical ingredient (API) effective to prevent, alleviate or ameliorate symptoms of disease or prolong the survival of the subject being treated. Determination of a therapeutically effective amount is well within the capability of those skilled in the art, especially in light of the detailed disclosure provided herein.

The term “subject” as used herein refers to an animal or a human. For instance, a subject in need of a treatment for an CNS disease can be a pediatric patient.

The term “treating” or “treatment” of any disease or condition refers, in some embodiments, to ameliorating the disease or disorder (i.e., arresting or reducing the development of the disease or at least one of the clinical symptoms thereof). In some embodiments “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both. In some embodiments, “treating” or “treatment” refers to delaying the onset of the disease or disorder, or even preventing the same. “Prophylactic treatment” is to be construed as any mode of treatment that is used to prevent progression of the disease or is used for precautionary purpose for persons at risk of developing the condition.

The term “impurity” or “total impurity” as used herein refers to the degradation product of the active ingredient. The amount of impurity can be determined by HPLC as illustrated in example 2. The total impurity may include impurity A and other impurities of unknown or uncharacterized structures.

The term “assay” used herein refers to the amount of an active ingredient. It can be determined with HPLC as shown in example 2.

The term “pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “therapeutically effective amount” refers to an amount of a compound effective to prevent, alleviate or ameliorate symptoms of disease or prolong the survival of the subject being treated. Determination of a therapeutically effective amount is well within the capability of those skilled in the art, especially in light of the detailed disclosure provided herein.

An aspect of the patent document provides a solid dosage composition containing API (vigabatrin) and optionally one or more pharmaceutically acceptable excipients. In some embodiments, the composition consists of the API.

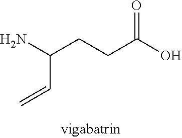

In some embodiments, the composition comprises API and at least one of sugar and sugar alcohol, and optionally a member selected from the group consisting of a sweetener, a preservative, a flavoring agent, a solubilizer, an antioxidant, a surfactant, an antifoaming agent, a chelating agent, a suspending agent, and a coloring agent or a combination thereof. In some embodiments, the solid dosage composition is in powder form. In some embodiments, the solid dosage composition remains stable with the impurity A no more than 1.0%, no more than 0.80%, no more than 0.60%, no more than 0.40%, no more than 0.20%, no more than 0.10%, no more than 0.08%, no more than 0.06% or no more than 0.05% within a certain period of time. In some embodiments, the solid dosage composition can be stable with the total impurity no more than 1.0%, no more than 0.80%, no more than 0.60%, no more than 0.40%, no more than 0.20%, no more than 0.10%, no more than 0.08%, no more than 0.06%, no more than 0.05% or no more than 0.04% within a certain period of time. The certain period of time may be 1 month, 2 months, 3 months, 6 months, 9 months, 12 months, 18 months, 24 months, 30 months, 36 months, 48 months, 60 months or any time between two of the aforementioned periods. In some embodiments, the solid dosage composition is free from a buffering agent. Total impurity includes impurity A and other impurities of unknown or uncharacterized structures. The structure of impurity A is shown below

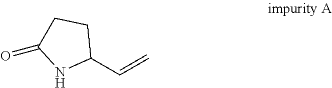

The liquid vehicle for reconstituting the solid composition into a solution contains one or more pharmaceutically acceptable excipients but is substantially free from the API. In some embodiments, the vehicle comprises at least water and a member selected from the group consisting of sweetener, preservative, buffering agent, flavoring agent, a solubilizer, antioxidant, surfactant, antifoaming agent, chelating agent, suspending agent, and coloring agent.

The sugar and sugar alcohol refer to but are not limited to one or more of sucrose, lactose, fructose, dextrose, mannitol, sorbitol and xylitol. The composition can contain any combination of sugar and/or sugar alcohol. The sugar and/or sugar alcohol in accordance with the present invention, can also function as a sweetener in the solid dosage composition for oral solution reconstitution. The amount of sugar and/or sugar alcohol used in the solid dosage composition of this present invention ranges from about 20% to about 99% w/w based on the total weight of the solid dosage composition. In some embodiments, the sugar and/or sugar alcohol used in the solid dosage composition ranges from about 20% to about 99%, from about 30% to about 99%, from about 40% to about 99%, from about 50% to about 99%, from about 60% to about 99%, from about 70% to about 99%, from about 80% to about 99%, from about 60% to about 90%, or from about 60% to about 80% of the total weight of the composition.

A buffering agent may be added in the vehicle. Examples of suitable buffer agents include but are not limited to: sodium citrate, citric acid, fumaric acid, tartaric acid, potassium citrate, sodium bicarbonate, potassium bicarbonate, sodium dihydrogen phosphate, disodium hydrogen phosphate, sodium hydroxide and potassium dihydrogen phosphate. The buffers should have sufficient capacity to remain in the desired pH range when reconstituting the powder for oral solution using water from different sources, which have different pH ranges. The buffer concentration after reconstituting ranges from about 10 mM to about 500 mM, preferably 10 mM to 400 mM, preferably 10 mM to 300 mM, preferably 10 mM to 200 mM, preferably 10 mM to 100 mM. In some embodiments, the buffer after reconstitution has a concentration of about 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 300 mM, 350 mM, 400 mM, 450 mM, 500 mM or a range before any two of the aforementioned values.

Preservatives may include benzalkonium chloride, cetalokonium chloride, benzoates (e.g. sodium benzoates), methyl paraben, ethyl paraben, propyl paraben, butylparaben, alkyl gallates, hydroxybenzoates and salts thereof (e.g. methyl or propyl hydroxybenzoates and salts thereof), phenyl mercuric salts (e.g. borates or nitrates), sodium hypocholorite, paraben, potassium sorbate, and sorbic acid. The preservative in some embodiments has a concentration after reconstituting ranging from about 0.02% (w/v) to about 10% (w/v), preferably 0.04% (w/v) to 5% (w/v), preferably 0.06% (w/v) to 4% (w/v), preferably 0.08% (w/v) to 3% (w/v), preferably 0.10% (w/v) to 2% (w/v), preferably 0.20% (w/v) to 1% (w/v). In some embodiments, the preservatives in the reconstituted solution are 0.02% (w/v), 0.04% (w/v), 0.06% (w/v), 0.08% (w/v), 0.10% (w/v), 0.12% (w/v), 0.14% (w/v), 0.16% (w/v), 0.18% (w/v), 0.20% (w/v), 0.22% (w/v), 0.24% (w/v), 0.26% (w/v), 0.28% (w/v) 0.30% (w/v), 0.32% (w/v), 0.34% (w/v), 0.36% (w/v), 0.38% (w/v), 0.40% (w/v) 0.42% (w/v), 0.44% (w/v), 0.46% (w/v), 0.48% (w/v), 0.50% (w/v), 0.60% (w/v), 0.70% (w/v), 0.80% (w/v) 0.90% (w/v), 1.0% (w/v) or a range before any two of the aforementioned values.

Sweeteners may include, for example, sucrose, glucose, sorbitol, sucralose, aspartame, saccharin sodium and any other pharmaceutically acceptable sweetener or combination thereof. The amount of the sweetener used in the powder for oral solution compositions is typically in the range of from about 0.1% to about 99%, preferably 0.1%-10% w/w based on the total weight of the powder for oral solution. In some exemplary embodiments, the sweetener is sucrose, glucose, sorbitol, sucralose, aspartame, saccharin sodium, any other pharmaceutically acceptable sweetener or any combination thereof.

The flavor may be selected from, for example, grenadine flavor, berry flavor, strawberry flavor, banana flavor, orange flavor and peppermint flavor. The amount of the flavor in the powder for oral solution is between about 0.1% to about 5% w/w based on the total weight of the powder for oral solution.

In the approved vigabatrin product(Vigafyde) including peppermint in the formulation. Peppermint flavor normally have peppermint oil. Side effects could happen if applying peppermint oil to the skin which include skin rashes and irritation. Peppermint oil should not be applied to the face of infants or young children because serious side effects may occur if they inhale the menthol in the oil. As the mucous membrane of infantile are not as perfect as human, the peppermint is irritated to their digestive tract, therefore medicate the drug containing peppermint flavor will have some safety concern. Thus, in some embodiment, the reconstituted solution of the invention is free of flavor. In some embodiment, the reconstituted solution of the invention is free of peppermint flavor.

The specifically mentioned suitable sugar, sugar alcohol, buffer, sweetener and flavor in the foregoing lists are intended to be exemplary, not exhaustive, of specific excipients that may be used in the practice of the disclosed invention. It is further understood that more than one of any particular type of excipient may be used in the powder for oral solution compositions described herein. For example, the compositions may include more than one sugar, sweetener, etc. Also, a single excipient may provide multiple functions.

Also provided herein is a kit containing the solid dosage composition in a first container and one or more excipients for storing or preparing a liquid vehicle in a second container. The scope and specifics of the solid dosage form and the excipients are as described above. The kit will also include instructions on reconstituting the composition and administering the solution.

This solid dosage for oral solution may be prepared by the general process known in the field. Examples include dry powder blending, wet granulation, dry granulation by compaction/slugging, spray drying, hot melt extrusion, extrusion spheronization and fluidized bed granulation. This solid dosage could be in the form of powder, blends, pellets, tablets, granules and other applicable solid form.

Generally, the solid form vigabatrin blends is stored in the first container prior to being mixed with a dissolving medium, for example water or vehicle, which is prepared or stored in a second container. In some embodiments, the first container contains only vigabatrin or its salt. The actual solution can be prepared by a pharmacist according to instructions for use for this solid dosage composition for oral solution. In an exemplary embodiment, a vehicle is prepared by adding accurate amount of water to the second container. The vehicle is then used to reconstitute the powder in the first container. Shaking can help to mix the water or vehicle with powder until all the solid is dissolved. The oral solution is thus the actual dosage form for multiple use by patients.

In some embodiments, the buffering agent is only present in the second container. The second container can certainly contain water in a pre-determined amount with the excipients dissolved therein so that the pharmacist or user does not need to add water to prepare the vehicle. In some embodiments, the buffering agent and the preservative are separately dissolved into water to prepare the vehicle. A syringe can be used to withdraw the appropriate dosing volume based on prescription for administration to the patient.

In some embodiments, impurity Ain the reconstituted solution is no more than 1.0%, no more than 0.80%, no more than 0.60%, no more than 0.40%, no more than 0.20%, no more than 0.10%, no more than 0.08%, no more than 0.06%. no more than 0.05%, no more than 0.04%, no more than 0.03%, no more than 0.02%, or no more than 0.01% as determined by HPLC within a certain period of time. In some embodiments, the total impurity in the reconstituted solution is no more than 1.0%, no more than 0.80%, no more than 0.60%, no more than 0.40%, no more than 0.20%, no more than 0.10%, no more than 0.08%, no more than 0.06%. no more than 0.05%, no more than 0.04%, no more than 0.03%, no more than 0.02%, or no more than 0.01% as determined by HPLC within a certain period of time. The certain period of time may be 1 month, 2 months, 3 months, 6 months, 9 months, 12 months, 18 months, 24 months, 30 months, 36 months, 48 months, 60 months or a time range between any two of the aforementioned time periods. With low levels of impurities and bacteria, the reconstituted solution is suitable for multiple uses over an extended period of time. In some embodiments, impurity A and/or the total impurity in the reconstituted solution are no more than 0.02% or no more than 0.01% as determined by HPLC within 3, 4 or 6 months after reconstitution.

After reconstituting the solid form into an oral solution, the liquid solution can provide easy and accurate dose titration for a subject to be treated, including for example pediatric patients and patients with dysphagia who cannot swallow tablets due to its size.

In some embodiments, the solid active ingredient for oral solution after reconstitution with vehicle provides a liquid solution containing about 500 mg of API per about 10 ml of liquid solution. The solid vigabatrin is stable on storage and when reconstituted with water for administration, the corresponding liquid solution is stable during the use in which the therapy is required. In some embodiments, the API concentration in the solution is above about 10 mg/ml. In some embodiments, the API concentration in the solution ranges from about 10 to about 100 mg/ml, from about 20 to about 100 mg/ml, from about 30 to about 100 mg/ml, from about 40 to about 100 mg/ml, from about 40 to about 90 mg/ml, from about 40 to about 80 mg/ml, from about 40 to about 70 mg/ml, or from about 40 to about 60 mg/ml.

The pH of the reconstituted solution ranges from about 5.0 to about 8.0. In some embodiments, the pH of the solution ranges from about 5.0 to about 7.0, from about 5.0 to about 6.0 or from about 6.0 to about 7.0. In some embodiments, the amount of the buffering agent is selected so that after reconstitution the pH of the solution is 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0 or a range between any two of the aforementioned values. In some embodiments, the amount of the buffering agent is selected so that after reconstitution the pH of the solution is about 5.5.

The sugar and/or sugar alcohol are present in the solution by 40-80% by weight. In some embodiments, the amount of the sugar and/or sugar alcohol in the solution ranges from about 50% to about 70%, from about 55% to about 65%, or from about 60% to about 70% by weight.

The paraben preservatives are present in this invention, In some embodiment, the propylparaben (sodium) to methylparaben (sodium) ratio should be no more than 1:100, preferably no more than 1:90, preferably no more than 1:80, preferably no more than 1:70, preferably no more than 1:60, preferably no more than 1:50, preferably no more than 1:40, preferably no more than 1:30, preferably no more than 1:20, preferably no more than 1:10, preferably no more than 1:9, preferably no more than 1:8, preferably no more than 1:7, preferably no more than 1:6, preferably no more than 1:5, preferably no more than 1:4, preferably no more than 1:3, preferably no more than 1:2 preferably no more than 1:1 or a range between any two of the aforementioned values.

The Antimicrobial Effectiveness Test (AET) is following USP<51> and performed by inoculating a test product with a known quantity of specified microorganisms, such as bacteria, yeasts, and molds. The quantity of microorganisms to inoculate to the test product at day 0 (initial inoculation moment) is then compared to the level of the microorganisms in the test product over specified intervals over a 14-day and 28-day period. In some embodiments, S. aureus, E. coli, P. aeruginosa and/or B. cepacia in the reconstituted solution at day 14 shows a reduction of not less than 0.1 log 10 unit, not less than 0.2 log 10 unit, not less than 0.4 log 10 unit, not less than 0.6 log 10 unit, not less than 0.8 log 10 unit, not less than 1.0 log 10 unit, not less than 1.2 log 10 unit, not less than 1.5 log 10 unit, not less than 2.0 log 10 unit, not less than 3.0 log 10 unit, not less than 4.0 log 10 unit, or not less than 5.0 log 10 unit from day 0, and/or no increase, or an increase of no more than 0.5 log 10 unit, no more than 0.4 log 10 unit, no more than 0.3 log 10 unit, no more than 0.2 log 10 unit, no more than 0.1 log 10 unit from the 14th day's count at 28 days, and/or wherein C. albicans and A. brasiliensis show no increase, or an increase of no more than 0.5 log 10 unit, no more than 0.4 log 10 unit, no more than 0.3 log 10 unit, no more than 0.2 log 10 unit, no more than 0.1 log 10 unit at day 14 and/or day 28 from day 0.

The reconstituted solution is suitable for multiple uses over an extended period of time. The quality of the solution remains acceptable during the entire period of the multiple uses. In some embodiments, the solution passes the Antimicrobial Effectiveness Test (AET) under USP <51> by the second, third, fifth, tenth, eleventh, fourteenth, twentieth, thirtieth, fortieth, fiftieth, sixtieth, eightieth, or ninetieth day since its preparation from the solid dosage composition described herein. The API in the solution maintains its efficacy during the entire period of the multiple uses, wherein at least about 70%, at least about 80%, at least about 85%, at least about 90%, at least about 95%, or at least about 98% of the API remains stable or undecomposed for multiple uses. In some embodiments, the API is vigabatrin.

Another aspect of the invention provides a method of treating a central nervous system (CNS) disease, comprising administering to a subject in need an oral solution described herein. The solution can be administered with an interval of at least about 30 minutes at least about 1 hour, at least about 5 hours, at least about 10 hours, at least 24 hours, at least 2 days, at least 5 days, at least 7 days, at least 15 days, at least 20 days, at least 30 days, at least 50 days, at least 60 days, or any combination of the above intervals. For example, after the initial administration, a second portion of the solution can be administered in 8 hours and a third portion can be administered in 24 hours after the second administration. A subsequent portion can be administered in a suitable interval. In some embodiments, the interval is at least 1-week long. In some embodiments, the interval ranges from about 1 week to about 3 months, from about 1 week to about 2 months or from about 2 weeks to about 2 months. The dose for each individual portion can be determined by one of ordinary skill in the art depending on the specific disease condition of the subject in need thereof. Of course, the container or kit of the composition will also provide guidance for a patient to administer the suitable amount of the solution. In some embodiments, the CNS disease is selected from the group consisting of infantile spasm, complex refractory seizure, tourette syndrome, refractory autoimmune encephalitis, cocaine dependence, preventing epilepsy in infants with tuberous sclerosis complex.

### EXAMPLE

The following examples are provided to enable one skilled in the art to practice the invention and are merely illustrative of the invention. The examples should not be read as limiting the scope of the invention as defined in the claims.

### Example 1: API Blends+Vehicle(Water)

For the multiple use product, the antimicrobial property should be guaranteed during the dosing period, so as to ensure the medication safety.

API and API blends (API+citric acid+phosphate) are reconstituted in water separately, then both were performed with Antimicrobial Effectiveness Test (AET) per USP<51>, the results are shown in table 1

TABLE 1
the comparison of AET result between API solution
and solution of API + buffer in HDPE bottle

| | | | Vigabatrin + citric acid/ | |
|---|---|---|---|---|
| | Vigabatrin + water | | phosphate + water | |
| Composition | 14D log | 28D log | 14D log | 28D log |
| AET | reduction | reduction | reduction | reduction |
| S. aureus | 1.8 | 4.7 | 0.7 | −0.5 |
| E. coli | 1.3 | 1.5 | −0.8 | −1.5 |
| P. aeruginosa | 2.3 | 3.6 | −1.6 | −1.9 |
| C. albicans | −0.3 | 0 | 2.9 | 3.9 |
| A. brasiliensis | 0.2 | 0.1 | 0.3 | 0.2 |
| Result | Pass | Pass | Fail | Fail |

These compositions are designated as category 3 products: Oral products other than antacids, made with aqueous bases or vehicles). The acceptance criteria for Tested Microorganisms is based on USP<51>. Specifically, the criteria includes the following.
Bacteria (S. aureus, E. coli, P. aeruginosa and B. cepacia): Not less than 1.0 log 10 unit reduction at day 14 from the initial count (day 0), and no increase at day 28 from day 14's count
Yeast and Molds (C. albicans and A. brasiliensis): No increase from the initial calculated count at day 14 and day 28.

No increase in microorganism counts is defined as not more than 0.5 log 10 unit between two time points (e.g. between day 0 and day 14 or between day 0 and day 28 or between day 14 and day 28).

Surprisingly, API+buffer group fail the AET due to the bacteria E. coli and P. aeruginosa increasing more than 0.5 log 10 unit count at day 14 day and day 28 day not meeting the criteria “NLT 1.0 log reduction from the initial count at 14 days, and no increase from the 14 days' count at 28 days”, while API group barely pass the test.

A new powder composition for preparation into an oral solution was made as described in Table 2 below.

TABLE 2
The powder composition for oral solution per 250 ml bottle

| Formulation 1 | g/250 ml | %(w/w) | Function |
|---|---|---|---|
| Vigabatrin | 12.5 | 5.6% | Active Pharmaceutical |
| | | | Ingredient |
| Sucrose | 212.5 | 94.4% | Diluent/Sweetener |

Vehicle (112.5 g water) was added into the powder to make up 250 ml solution in a bottle. The bottle was shaken intensely until all the solid dissolved and formed a colorless and clear solution with the concentration of 50 mg/ml.

Take the sole API as control, both the reconstituted API solution and solution of API+sucrose blends have been tested for Antimicrobial Effectiveness Test(AET) per USP<51> plus B. cepacian which is recommended by regulatory guideline for the liquid product. Each solution has been conducted on two different bottles.

Surprisingly, the reconstituted solution of the novel powder composition all passed the AET that have log reduction more than 1 log 10 unit with different bottles. The only API solution that failed at day 14 day is due to B. cepacian bacteria with only 0.8 log 10 unit reduction in HJDPE bottle and 0.5 log 10 unit reduction in PET bottle count at day 14 not meeting the requirement “NLT 1.0 log reduction from the initial count at day 14”. The results are exhibited in Table 3 and Table 4.

TABLE 3
the comparison of AET result between API solution
and API + sucrose solution in HDPE bottle

| | Day 7 | | Day 14 | | Day 28 | |
|---|---|---|---|---|---|---|
| | Log Reduction | | Log Reduction | | Log Reduction | |
| | | API + | | API + | | API + |
| Test | API | sucrose | API | sucrose | API | sucrose |
| Organism | solution | solution | solution | solution | solution | solution |
| S. aureus | NA | 1.6 | 2.8 | 3.4 | >4.7 | >4.7 |
| E. coli | NA | 1.9 | 3.1 | 2.9 | >4.8 | >4.7 |
| P. aeruginosa | NA | >4.7 | 3.2 | >4.7 | 3.2 | >4.7 |
| B. cepacia | NA | >4.6 | 0.8 | >4.6 | 1.0 | >4.6 |
| C. albicans | NA | 2.7 | 0.7 | 4.1 | 0.5 | >4.4 |
| A. brasiliensis | NA | 0.8 | 0.9 | 1.1 | 0.7 | 0.5 |

TABLE 4
the comparison of AET result between API solution
and API + sucrose solution in PET bottle

| | Day 7 | | Day 14 | | Day 28 | |
|---|---|---|---|---|---|---|
| | Log Reduction | | Log Reduction | | Log Reduction | |
| | | API + | | API + | | API + |
| Test | API | sucrose | API | sucrose | API | sucrose |
| Organism | solution | solution | solution | solution | solution | solution |
| S. aureus | NA | 2.8 | 3.0 | >4.7 | >4.7 | >4.7 |
| E. coli | NA | 2.6 | 2.4 | 2.8 | 3.4 | 4.4 |
| P. aeruginosa | NA | >4.7 | 3.5 | >4.7 | 2.7 | >4.7 |
| B. cepacia | NA | >4.6 | 0.5 | >4.6 | 1.5 | >4.6 |
| C. albicans | NA | 2.8 | 0.6 | 3.2 | 0.5 | >4.4 |
| A. brasiliensis | NA | 0.9 | 0.8 | 1.3 | 0.6 | 0.8 |

The data showed API powder combine with water alone can't pass the ALT due to B. cepacian. The vehicle (water+excipients) was further investigated. The preservatives and other excipients were added in the vehicle and then reconstitute the API into solution. The formulation and results were presented in table 5.

TABLE 5
Two formulation with different preservatives

| | | Formulation | Formulation |
|---|---|---|---|
| Ingredients | Function | 2 | 3 |

Power Composition

| | | | |
|---|---|---|---|
| Vigabatrin | API | 24.857 | 25.115 |

Vehicle composition

| | | | |
|---|---|---|---|
| xanthan gum | Suspending | 0.996 | NA |
| | agent | | |
| Saccharin sodium | Sweetener | 0.099 | NA |
| Sucralose | | NA | 0.100 |
| Sodium citrate dihydrate | Buffer | 0.448 | 0.452 |
| Critic acid | | 0.597 | 1.255 |
| Sodium propylparaben | Preservative | NA | 0.068 |
| Propylparaben | | 0.040 | NA |
| Potassium sorbate | | 0.500 | 0.753 |
| Sodium methylparaben | | NA | 0.384 |
| Methylparaben | | 0.150 | NA |
| Purified water | Solvent | 477.170 | 477.013 |
| Subtotal | NA | 480.00 | 480.00 |

| | | |
|---|---|---|
| Total | 500 ml | 500 ml |
| AET | Fail | Pass |

The data showed a specific amount or ratio of the preservatives in the formulation could control microorganism growth in the solution to pass the AET test. The paraben amount in the vehicle should be NLT 0.040% (w/v) and preferably the propylparaben (sodium) to methylparaben (sodium) is no more than 1:5.

### Example 2 API (Vigabatrin)+Vehicle Stability

Prepare the API aqueous solution with two different concentration 50 mg/ml and 100 mg/ml and stored at the 40° C./75% RH for 3M as control. The assay and impurity of API aqueous solution were tested by HPLC method separately and the HPLC method list as below.

### HPLC Method for Assay:

Column: Partisil 10 SCX, 4.6×250 mm
Mobile Phase: Acetonitrile: Methanol: Buffer=4:40:1000(v:v:v)
UV detector: 210 nm
Flow rate: 1.5 ml/min
Injection volume: 20 μL
Column Temperature: 25° C.
Autosampler Temperature: Ambient
Run time: 15 minutes
Elution program: Isocratic

### HPLC Method for Impurity:

Column: Welch Ultimate AQ C18, 4.6×300 mm
Mobile Phase A: 1.5 g/L ammonium acetate Mobile Phase B: Acetonitrile
UV detector: 210 nm
Flow rate: 1.0 ml/min
Injection volume: 20 μL
Column Temperature: 30° C.
Autosampler Temperature: Ambient
Run time: 45 minutes
Elution program:

| Time(min) | MPA(%) | MPB(%) |
|---|---|---|
| 0 | 100 | 0 |
| 6 | 100 | 0 |
| 9 | 90 | 10 |
| 20 | 90 | 10 |
| 21 | 55 | 45 |
| 35 | 55 | 45 |
| 35.1 | 100 | 0 |
| 45 | 100 | 0 |

The stability data tested by the above HPLC method were presented in table 6 and table 7.

TABLE 6
The stability of API aqueous solution with
50 mg/ml stored under 40° C./75% RH for 3 M

| 20231219-4 | | | | |
|---|---|---|---|---|
| (40° C./75% RH) | 0 | 1 month | 2 month | 3 month |
| pH | 6.82 | 7.03 | 6.95 | 7.06 |
| assay | 98.5% | 99% | 98.6% | 100% |
| Total unknown impurity(%) | ND | ND | ND | ND |
| Impurity A(%) | ND | <LOQ | 0.07 | 0.09 |
| Total impurity(%) | ND | □ LOQ | 0.07 | 0.09 |

Note:
ND not detected,
N/A not applicable

TABLE 7
The stability of API aqueous solution with
100 mg/ml stored under 40° C./75% RH for 3 M

| 20231219-5 | | | | |
|---|---|---|---|---|
| (40° C./75% RH) | 0 [symbol] | 1 month | 2 month | 3 month |
| pH | 7.00 | 7.04 | 6.96 | 7.05 |
| assay | 98.7 | 100.2 | 98.8 | 100.3 |
| Total unknown impurity (%) | ND | ND | ND | ND |
| Impurity A(%) | ND | <LOQ | 0.08 | 0.11 |
| Total impurity(%) | ND | ND | 0.08 | 0.11 |

Both API aqueous solution with 50 mg/ml and 100 mg/ml showing obvious impurity increase under high temperature storage, which may have quality and safety risk before dispensing to the patient.

A powder composition and vehicle composition for preparation into an oral solution was made as described in Table 8 below.

TABLE 8
The powder composition and vehicle composition per 500 ml bottle

| | |
|---|---|
| Ingredients | Function |

Power Composition

| | | |
|---|---|---|
| Vigabatrin | 25 | Active Pharmaceutical |
| | | Ingredient |

Vehicle composition

| | | |
|---|---|---|
| sodium methylparaben | 0.300-0.700 | Preservative |
| sodium propylparaben | 0.060-0.120 | Preservative |
| Sodium citrate dihydrate | 0.300-0.600 | Buffer |
| Citric acid | 1.000-1.500 | Buffer |
| Sucralose | 0.080-0.120 | Sweetener |
| Water | q.s to 480 ml | Solvent |

| | |
|---|---|
| Total | 500 ml |

Each composition was placed in a glass bottle and stored at the 40° C./75% RH for 3 and 6 month to check its stability. The bottles of vehicle were placed in two different position upright and reverse. The stability data was presented as table 9 and table 10.

TABLE 9
Powder composition stability storage at 40° C./75% RH.

| Lot 2401004 | | | | |
|---|---|---|---|---|
| Testing item | | 0 | 3 month | 6 month |
| Appearance | | White to | White to | White to |
| | | off white | off white | off white |
| | | powder | powder | powder |
| LOD | | 0.8% | 0.1% | 0.1% |
| Assay | | 99.8% | 99.9% | 100.1% |
| Related | Impurity A | ND | ND | ND |
| substance | Unspecified | ND | ND | ND |
| | impurity | | | |
| | Total | ND | ND | ND |
| | impurity | | | |

| | | | |
|---|---|---|---|
| Microbial limit test | Comply with | N/A | Comply with |
| | USP<61>, | | USP<61>, |
| | <62> | | <62> |

TABLE 10
vehicle composition stability storage at 40° C./75% RH

| Lot 2401001 | | 3 Month | 3 Month | 6 Month | 6 Month |
|---|---|---|---|---|---|
| Testing item | 0 | (upright) | (reverse) | (upright) | (reverse) |
| Appearance | Colorless | Colorless | Colorless | Colorless | Colorless |
| sodium | 98.2% | 97.4% | 97.1% | 97% | 97% |
| methylparaben | | | | | |
| content | | | | | |
| sodium propyl | 100.5% | 98.1% | 97.7% | 98% | 98% |
| paraben content | | | | | |
| Antimicrobial | Comply with | N/A | N/A | N/A | Comply with |
| Effectiveness Test | USP<51> | | | | USP<51> |
| Microbial Limit | Comply with | N/A | N/A | Comply with | Comply with |
| Testing | USP<61>, <62> | | | USP<61>, <62> | USP<61>, <62> |

Each composition was well within spec and showed good stability. Considering the patient was dosed with reconstituted solution, after each composition stored at 40° C./75% RH for 6 month, then 480 mL Vehicle was added into the powder to make up 500 ml solution in a bottle. The bottle was shaken intensely until all the solid dissolved and formed a colorless and clear solution with the concentration of 50 mg/ml. The resultant solution was checked the in-use stability under ambient conditions, which was shown in table 11.

TABLE 11
in-use 3 month stability under 25° C./60% RH of the
reconstituted solution by powder composition and vehicle
composition after storge for 6 month under 40° C./75% RH

| Lot 2401004 + lot 2401001 | | | |
|---|---|---|---|
| Testing item | | 0 | 3 month |
| Appearance | | Colorless, no | Colorless, no |
| | | visible particle | visible particle |
| pH | | 5.4 | 5.5 |
| sodium methylparaben content | | 96% | 97% |
| sodium propyl paraben content | | 98% | 98% |
| Related | Impurity A | ND | <LOQ |
| substance | Unspecified | <LOQ | ND |
| | impurity | | |
| | Total impurity | <LOQ | <LOQ |

| | | |
|---|---|---|
| Assay | 105.1% | 104.6% |
| Microbial Limit Testing | Comply with | Comply with |
| | USP<61>, | USP<61>, |
| | <62> | <62> |

Per Patent US120168571B2, the Vigabatrin solution were stored under 40° C. for 3M, then its impurity A grew up to 0.21%, and the total impurity became 0.22%, while our kit design show both impurity A and total impurity not detected under 40° C. for 3M and even 6M. After 6M storage under high temperature, the resultant vigabatrin solution reconstituted by powder composition and vehicle still present desirable stability for 3M under 25° C./60% RH which was comparable or superior to the stability data of the solution only stored under 25° C./60% RH for 3M in Patent U.S. Ser. No. 12/016,857B2. Considering if the Vigabatrin solution were prepared and stored for some time before dispensing to patient and dosing, the impurity could increase obviously especially experienced high temperature before caregiver give medication to the patient while not releasing this, that could have potential quality concern, ultimately safety issue. While our unique kit design powder in one bottle and vehicle in another bottle not only ensures the product quality during storage especially under high temperature, but also can provide the qualified reconstitution solution when ready to dose, and even for multiple use need during a certain period after reconstitution.

### Example 3 API (Vigabatrin)+Vehicle at Different pH

The literature show the aqueous solution of paraben preservative at pH 3-6 are stable, while different pH of reconstituted solution were investigated, surprisingly, only pH5.5 can fit for the Vigabatrin solution which have good stability data. Therefore a designated pH range with pH5.0-8.0, preferably 5.0-7.0 are selected for the patent. The results are shown in table 12 to table 15.

TABLE 12
the stability of reconstituted solution of powder composition
and vehicle at pH 2.5 under 25° C./60% RH

| 20231219-3 pH 2.5 | | | | |
|---|---|---|---|---|
| (25° C./60% RH) | 0 | 1 month | 2 month | 3 month |
| pH | 2.55 | 2.65 | 2.64 | 2.62 |
| Assay (%) | 95.8 | 96.3 | 95.8 | 96.8 |
| Total unknown impurity (%) | 0.21 | 0.11 | 0.11 | 0.14 |
| Impurity A (%) | 0.01 | 0.07 | 0.14 | 0.19 |
| Total impurity (%) | 0.22 | 0.19 | 0.25 | 0.33 |

TABLE 13
the stability of reconstituted solution of powder composition
and vehicle at pH 3.5 under 25° C./60% RH

| 20231219-2 pH 3.5 | | | | |
|---|---|---|---|---|
| (25° C./60% RH) | 0 | 1 month | 2 month | 3 month |
| pH | 3.59 | 3.65 | 3.62 | 3.62 |
| Assay (%) | 99.4 | 100 | 99.2 | 100.4 |
| Total unknown impurity (%) | ND | ND | ND | ND |
| Impurity A (%) | 0.01 | 0.08 | 0.16 | 0.21 |
| Total impurity (%) | 0.1 | 0.08 | 0.16 | 0.21 |

TABLE 14
the stability of reconstituted solution of powder composition
and vehicle at pH 4.5 under 25° C./60% RH

| 20231219-1 pH 4.5 | | | | |
|---|---|---|---|---|
| (25° C./60% RH) | 0 | 1 month | 2 month | 3 month |
| pH | 4.6 | 4.61 | 4.59 | 4.6 |
| Assay (%) | 99.5 | 100.2 | 99.8 | 100.4 |
| Total unknown impurity (%) | ND | ND | ND | ND |
| Impurity A (%) | 0.01 | 0.05 | 0.1 | 0.14 |
| Total impurity (%) | 0.1 | 0.05 | 0.1 | 0.14 |

TABLE 15
the stability of reconstituted solution of powder composition
and vehicle at pH 5.5 under 25° C./60% RH

| 20220602Ab pH 5.5 | | | | |
|---|---|---|---|---|
| (25° C./60% RH) | 0 | 1 month | 2 month | 3 month |
| pH | 5.34 | 5.41 | 5.44 | 5.46 |
| Assay (%) | 100.5 | 99.4 | 101.3 | 100.1 |
| Total unknown impurity (%) | ND | ND | ND | ND |
| Impurity A (%) | ND | <LOQ | 0.035 | 0.059 |
| Total impurity (%) | ND | ND | 0.035 | 0.059 |

### Example 4 Dosing Kit

Two dosing syringe 3 ml and 10 ml were supplied with the kit when ready for dosing, that could provide accurate dose especially for the pediatric drug requiring dose adjustment based on the body weight. The bottle of powder and the bottle of vehicle can be stored at its original form. When ready for dosing, the powder and the vehicle are mixed to prepare a reconstituted solution, from which as desirable dose can be withdrawn with provided syringes. An adaptor can be pressed in the bottle neck after reconstitution to facilitate the withdrawal of the solution with the syringes.

The accurate dosing of two syringe were investigated, the data are shown in table 16 to table 22

For 3 mL syringes: a series weighing of delivered amount of 1.5 mL and 3 mL were performed. The desired delivered amount from one syringe was evaluated each time at each use for 50 consecutive uses. The desired delivered amounts for two other 3 mL syringes were evaluated at the selected uses of Use 1, Use 10, Use 30 and Use 50 for 50 consecutive uses.

For 10 mL syringes: a series weighing of delivered amount of 3.5 mL, 5 mL and 10 mL were performed. The desired delivered amount from one syringe was evaluated each time at each use for 50 consecutive uses. The desired delivered amount for two other 10 mL syringes were evaluated at the selected uses of Use1, Use 10, Use 30 and Use 50 for 50 consecutive uses.

Density of reconstituted solution of reconstituted Vigabatrin Solution was measured in accordance to USP <841> Specific Gravity Method I. Volume from each delivery of the desired amount of each use from the syringes was obtained from the calculation: Volume=Weight/Density.

TABLE 16
Data obtained from 3 mL syringe #1 with target of 1.5 mL delivery

| # of | Volume | # of | Volume | # of | Volume | # of | Volume | # of | Volume |
|---|---|---|---|---|---|---|---|---|---|
| withdraw | (mL) | withdraw | (mL) | withdraw | (mL)/ | withdraw | (mL) | withdraw/ | (mL) |
| 1 | 1.53 | 11 | 1.51 | 21 | 1.51 | 31 | 1.51 | 41 | 1.50 |
| 2 | 1.52 | 12 | 1.51 | 22 | 1.50 | 32 | 1.49 | 42 | 1.53 |
| 3 | 1.51 | 13 | 1.50 | 23 | 1.50 | 33 | 1.50 | 43 | 1.50 |
| 4 | 1.50 | 14 | 1.49 | 24 | 1.51 | 34 | 1.50 | 44 | 1.50 |
| 5 | 1.52 | 15 | 1.50 | 25 | 1.50 | 35 | 1.50 | 45 | 1.51 |
| 6 | 1.50 | 16 | 1.51 | 26 | 1.50 | 36 | 1.51 | 46 | 1.50 |
| 7 | 1.51 | 17 | 1.50 | 27 | 1.51 | 37 | 1.50 | 47 | 1.50 |
| 8 | 1.51 | 18 | 1.50 | 28 | 1.50 | 38 | 1.49 | 48 | 1.50 |
| 9 | 1.51 | 19 | 1.51 | 29 | 1.51 | 39 | 1.51 | 49 | 1.50 |
| 10 | 1.52 | 20 | 1.50 | 30 | 1.49 | 40 | 1.51 | 50 | 1.50 |

| | | | | | | | |
|---|---|---|---|---|---|---|---|
| Max value | 1.53 | Min value | 1.49 | Mean value | 1.51 | RSD/% | 0.51 |

TABLE 17
Data obtained from 3 mL syringe #1 with target of 3 mL delivery

| # of | Volume | # of | Volume | # of | Volume | # of | Volume | # of | Volume |
|---|---|---|---|---|---|---|---|---|---|
| withdraw | (mL) | withdraw | (mL) | withdraw | (mL)/ | withdraw | (mL) | withdraw/ | (mL) |
| 1 | 2.97 | 11 | 2.97 | 21 | 2.96 | 31 | 2.97 | 41 | 2.99 |
| 2 | 2.96 | 12 | 2.95 | 22 | 2.98 | 32 | 2.97 | 42 | 2.98 |
| 3 | 2.97 | 13 | 2.98 | 23 | 2.97 | 33 | 2.98 | 43 | 2.98 |
| 4 | 2.95 | 14 | 2.96 | 24 | 2.96 | 34 | 2.97 | 44 | 2.99 |
| 5 | 2.96 | 15 | 2.97 | 25 | 2.97 | 35 | 2.97 | 45 | 2.98 |
| 6 | 2.98 | 16 | 2.98 | 26 | 2.97 | 36 | 2.98 | 46 | 2.97 |
| 7 | 2.97 | 17 | 2.97 | 27 | 2.97 | 37 | 2.97 | 47 | 2.97 |
| 8 | 2.97 | 18 | 2.97 | 28 | 2.98 | 38 | 2.99 | 48 | 2.96 |
| 9 | 2.97 | 19 | 2.99 | 29 | 2.98 | 39 | 2.97 | 49 | 2.98 |
| 10 | 2.99 | 20 | 2.98 | 30 | 2.97 | 40 | 2.98 | 50 | 2.96 |

| | | | | | | | |
|---|---|---|---|---|---|---|---|
| Max value | 2.99 | Min value | 2.95 | Mean value | 2.97 | RSD/% | 0.31 |

TABLE 18
Data obtained from 3 mL syringe #2 & 3 and all
data statistics of syringe #1 & 2 & 3

| 3 mL #2 | | 3 mL #3 | | 3 mL #2 | | 3 mL #3 | |
|---|---|---|---|---|---|---|---|
| 1.5 mL delivered | | 1.5 mL delivered | | 3 mL delivered | | 3 mL delivered | |
| # of | Volume | # of | Volume | # of | Volume | # of | Volume |
| withdraw | (mL) | withdraw | (mL) | withdraw | (mL) | withdraw | (mL) |
| 1 | 1.50 | 1 | 1.49 | 1 | 2.99 | 1 | 2.98 |
| 10 | 1.49 | 10 | 1.49 | 10 | 3.00 | 10 | 2.99 |
| 30 | 1.50 | 30 | 1.50 | 30 | 2.98 | 30 | 2.98 |
| 50 | 1.51 | 50 | 1.48 | 50 | 2.99 | 50 | 2.99 |
| Mean value | 1.50 | RSD/% | 0.57 | Mean value | 2.97 | RSD/% | 0.34 |
| Max value | 1.53 | Min value | 1.48 | Max value | 3.00 | Min value | 2.95 |

TABLE 19
Data obtained from 10 mL syringe #1 with target of 3.5 mL delivery

| # of | Volume | # of | Volume | # of | Volume | # of | Volume | # of | Volume |
|---|---|---|---|---|---|---|---|---|---|
| withdraw | (mL) | withdraw | (mL) | withdraw | (mL)/ | withdraw | (mL) | withdraw/ | (mL) |
| 1 | 3.51 | 11 | 3.46 | 21 | 3.48 | 31 | 3.47 | 41 | 3.49 |
| 2 | 3.50 | 12 | 3.49 | 22 | 3.48 | 32 | 3.45 | 42 | 3.50 |
| 3 | 3.47 | 13 | 3.46 | 23 | 3.45 | 33 | 3.48 | 43 | 3.49 |
| 4 | 3.46 | 14 | 3.49 | 24 | 3.48 | 34 | 3.51 | 44 | 3.50 |
| 5 | 3.47 | 15 | 3.49 | 25 | 3.48 | 35 | 3.51 | 45 | 3.49 |
| 6 | 3.45 | 16 | 3.47 | 26 | 3.46 | 36 | 3.49 | 46 | 3.48 |
| 7 | 3.51 | 17 | 3.46 | 27 | 3.45 | 37 | 3.51 | 47 | 3.49 |
| 8 | 3.48 | 18 | 3.47 | 28 | 3.47 | 38 | 3.49 | 48 | 3.49 |
| 9 | 3.50 | 19 | 3.48 | 29 | 3.49 | 39 | 3.48 | 49 | 3.49 |
| 10 | 3.50 | 20 | 3.47 | 30 | 3.47 | 40 | 3.51 | 50 | 3.47 |

| | | | | | | | |
|---|---|---|---|---|---|---|---|
| Max value | 3.51 | Min value | 3.45 | Mean value | 3.48 | RSD/% | 0.52 |

TABLE 20
Data obtained from 10 mL syringe #1 with target of 5 mL delivery

| # of | Volume | # of | Volume | # of | Volume | # of | Volume | # of | Volume |
|---|---|---|---|---|---|---|---|---|---|
| withdraw | (mL) | withdraw | (mL) | withdraw | (mL)/ | withdraw | (mL) | withdraw/ | (mL) |
| 1 | 4.96 | 11 | 4.97 | 21 | 4.98 | 31 | 4.98 | 41 | 5.00 |
| 2 | 4.96 | 12 | 4.96 | 22 | 4.99 | 32 | 4.96 | 42 | 4.95 |
| 3 | 4.96 | 13 | 4.98 | 23 | 4.96 | 33 | 4.97 | 43 | 4.97 |
| 4 | 4.96 | 14 | 4.97 | 24 | 4.95 | 34 | 4.96 | 44 | 4.95 |
| 5 | 4.97 | 15 | 4.98 | 25 | 4.96 | 35 | 5.00 | 45 | 4.96 |
| 6 | 4.99 | 16 | 4.99 | 26 | 4.95 | 36 | 4.98 | 46 | 4.95 |
| 7 | 4.95 | 17 | 4.96 | 27 | 4.96 | 37 | 4.95 | 47 | 4.98 |
| 8 | 4.97 | 18 | 4.98 | 28 | 4.96 | 38 | 4.98 | 48 | 4.96 |
| 9 | 4.99 | 19 | 4.98 | 29 | 4.97 | 39 | 4.98 | 49 | 4.96 |
| 10 | 4.97 | 20 | 4.95 | 30 | 4.96 | 40 | 4.96 | 50 | 4.96 |

| | | | | | | | |
|---|---|---|---|---|---|---|---|
| Max value | 5.00 | Min value | 4.95 | Mean value | 4.97 | RSD/% | 0.28 |

TABLE 21
Data obtained from 10 mL syringe #1 with target of 10 mL delivery

| # of | Volume | # of | Volume | # of | Volume | # of | Volume | # of | Volume |
|---|---|---|---|---|---|---|---|---|---|
| withdraw | (mL) | withdraw | (mL) | withdraw | (mL)/ | withdraw | (mL) | withdraw/ | (mL) |
| 1 | 9.99 | 11 | 9.95 | 21 | 9.98 | 31 | 10.00 | 41 | 9.99 |
| 2 | 9.98 | 12 | 10.02 | 22 | 10.00 | 32 | 9.99 | 42 | 9.96 |
| 3 | 10.01 | 13 | 10.03 | 23 | 10.00 | 33 | 10.03 | 43 | 10.00 |
| 4 | 10.02 | 14 | 10.00 | 24 | 9.98 | 34 | 9.99 | 44 | 9.98 |
| 5 | 9.99 | 15 | 9.99 | 25 | 9.98 | 35 | 9.99 | 45 | 10.02 |
| 6 | 10.00 | 16 | 9.98 | 26 | 9.99 | 36 | 10.00 | 46 | 9.96 |
| 7 | 9.99 | 17 | 10.02 | 27 | 10.03 | 37 | 9.99 | 47 | 9.99 |
| 8 | 9.99 | 18 | 9.99 | 28 | 10.00 | 38 | 9.98 | 48 | 9.98 |
| 9 | 9.97 | 19 | 9.96 | 29 | 9.99 | 39 | 9.98 | 49 | 10.01 |
| 10 | 10.01 | 20 | 9.99 | 30 | 9.99 | 40 | 9.99 | 50 | 9.99 |

| | | | | | | | |
|---|---|---|---|---|---|---|---|
| Max value | 10.03 | Min value | 9.95 | Mean value | 9.99 | RSD/% | 0.17 |

TABLE 22
Data obtained from 10 mL syringe #2 & 3 and data statistics of syringe #1 & 2 & 3

| 10 mL #2 | | 10 mL #3 | | 10 mL #2 | | 10 mL #3 | | 10 mL #2 | | 10 mL #3 | |
|---|---|---|---|---|---|---|---|---|---|---|---|
| 3.5 mL delivered | | 3.5 mL delivered | | 5 mL delivered | | 5 mL delivered | | 10 mL delivered | | 10 mL delivered | |
| # of | Volume | # of | Volume | # of | Volume | # of | Volume | # of | Volume | # of | Volume |
| withdraw | (mL) | withdraw | (mL) | withdraw | (mL) | withdraw | (mL) | withdraw | (mL) | withdraw | (mL) |
| 1 | 3.49 | 1 | 3.51 | 1 | 4.96 | 1 | 5.00 | 1 | 9.99 | 1 | 10.02 |
| 10 | 3.46 | 10 | 3.49 | 10 | 4.98 | 10 | 4.99 | 10 | 10.00 | 10 | 9.98 |
| 30 | 3.51 | 30 | 3.47 | 30 | 5.00 | 30 | 5.00 | 30 | 9.99 | 30 | 9.99 |
| 50 | 3.50 | 50 | 3.47 | 50 | 4.97 | 50 | 4.98 | 50 | 9.98 | 50 | 10.02 |
| Mean value | 3.48 | RSD/% | 0.51 | Mean value | 4.97 | RSD/% | 0.32 | Mean value | 9.99 | RSD/% | 0.17 |
| Max value | 3.51 | Min value | 3.45 | Max value | 5.00 | Min value | 4.95 | Max value | 10.03 | Min value | 9.95 |

The data prove that after multiple withdrawal of vigabatrin solution, the supplied dosing syringe could still provide the accurate volume, especially for low dose which should guarantee the medication accuracy for the infantile and pediatrics.

1. A kit for storing a solid form of vigabatrin or a pharmaceutically acceptable salt thereof and reconstituting it to a solution, comprising
(a) a first container containing the solid form of vigabatrin or a pharmaceutically acceptable salt thereof, wherein the first container is free from a buffering agent and a preservative;
(b) a second container for storing or preparing a vehicle for reconstituting the solid form of vigabatrin, wherein the second container contains water, a buffering agent, a sweetener, and a preservative;
wherein the preservative is completely dissolved in water and comprises methylparaben or a salt thereof, propylparaben or a salt thereof, or a combination thereof,
wherein the preservative is present in an amount ranging from about 0.04% (w/v) to about 10% (w/v),
wherein the buffering agent comprises sodium citrate dihydrate, citric acid, or a combination thereof;
wherein after reconstitution total impurity in the solution is no more than 0.04% within 2 months at room temperature, wherein the solution has a pH ranging from 5.3 to 6.0, and
wherein, according to Antimicrobial Effectiveness Test (AET) under USP <51>, S. aureus, E. coli, P. aeruginosa and/or B. cepacia in the reconstituted solution at day 14 shows not less than 1.0 log 10 unit reduction from day 0, and/or no increase in log 10 unit at day 28 from day 14, and/or wherein C. albicans and A. brasiliensis show no increase in log 10 unit at day 28 from day 0.

2. (canceled)

3. (canceled)

4. The kit of claim 1, wherein the buffering agent after reconstitution has a concentration ranging from about 10 mM to about 500 mM.

5. The kit of claim 1, wherein the preservative in the second container is in an amount so that after reconstitution the preservative has a concentration ranging from about 0.06% (w/v) to about 1% (w/v).

6. The kit of claim 1, wherein the buffering agent, the sweetener, and the preservative, and their respective amounts in the kit are selected so that, after reconstitution, S. aureus, E. coli, and P. aeruginosa in the solution at day 14 show not less than 1.0 log 10 unit reduction from day 0, and no increase in log 10 unit at day 28 from day 14, and wherein C. albicans and A. brasiliensis in the solution show no increase in log 10 unit at day 28 from day 0.

7. The kit of claim 6, wherein the buffering agent, the sweetener, and the preservative, and their respective amounts in the kit are selected so that, after reconstitution, B. cepacia in the solution at day 14 shows not less than 1.0 log 10 unit reduction from day 0, and no increase in log 10 unit at day 28 from day 14.

8. The kit of claim 1, wherein after reconstitution the solution is suitable for at least two separate administrations having an interval of at least 30 days.

9. The kit of claim 1, wherein the amounts of the solid form of vigabatrin or a pharmaceutically acceptable salt thereof and the vehicle are selected so that, after reconstitution, the concentration of the vigabatrin or a pharmaceutically acceptable salt thereof in the solution ranges from about 40 to about 60 mg/ml.

10. The kit of claim 1, further comprising a syringe for the solution after reconstitution.

11. (canceled)

12. The kit of claim 1, wherein the preservative further comprises one or more of compounds selected from the group consisting of benzalkonium chloride, cetalokonium chloride, benzoate, methylparaben or salt thereof, ethyl paraben or a salt thereof, propylparaben or a salt thereof, butylparaben or a salt thereof, alkyl gallate, hydroxybenzoate or salt thereof, phenyl mercuric salt, sodium hypochlorite, paraben, potassium sorbate, sorbic acid, and any combination thereof.

13. (canceled)

14. The kit of claim 1, wherein the buffering agent further comprises one or more of compounds selected from the group consisting of sodium citrate, citric acid, fumaric acid, tartaric acid, potassium citrate, sodium bicarbonate, potassium bicarbonate, sodium dihydrogen phosphate, disodium hydrogen phosphate, sodium hydroxide, potassium dihydrogen phosphate, and any combination thereof.

15. The kit of claim 1, wherein after reconstitution impurity A in the solution is 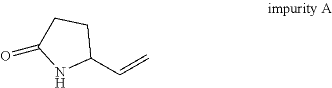
below 0.1% for 6 months at room temperature.

16. The kit of claim 1, wherein after reconstitution the total impurity in the solution is below 0.1% for 6 months at room temperature.

17. A method of treating a central nervous system (CNS) disease in a subject, comprising
(a) providing a kit of claim 1 and reconstituting the solid form of vigabatrin or a pharmaceutically acceptable salt thereof with the vehicle to obtain the solution;
(b) withdrawing a therapeutically effective amount of vigabatrin or a pharmaceutically acceptable salt thereof from the solution; and
(c) administering the therapeutically effective amount of vigabatrin or a pharmaceutically acceptable salt thereof to the subject in need thereof.

18. The method of claim 17, wherein the disease is selected from the group consisting of infantile spasm, complex refractory seizure, tourette syndrome, refractory autoimmune encephalitis, cocaine dependence, and epilepsy in infants with tuberous sclerosis complex.

19. The method of claim 17, which is applied to the subject prophylactically.

20. The method of claim 17, wherein step (c) is repeated at least twice with an interval of at least 4-6 hours.

21. The kit of claim 1, wherein the first container contains sucrose.

22. The kit of claim 1, wherein the pH of the solution ranges from 5.3 to 5.7.
